(19) United States
(12) Patent Application Publication
(10) Pub. No.: US 2012/0282645 A1
(43) Pub. Date: Nov. 8, 2012

(54) NANOPARTICLES

(75) Inventors: Superb K. Misra, London (GB); Agnieszka Dybowska, London (GB); Deborah Berhanu, London (GB); Eugenia Valsami-Jones, London (GB)

(73) Assignee: NATURAL HISTORY MUSEUM, London (GB)

(21) Appl. No.: 13/458,097

(22) Filed: Apr. 27, 2012

Related U.S. Application Data
(60) Provisional application No. 61/479,753, filed on Apr. 27, 2011.

(51) Int. Cl. A61K 33/34; C01G 9/02; A61K 33/30; B32B 5/16; C01G 3/02; G01N 27/62; B82Y 40/00; B82Y 30/00

(52) U.S. Cl. 435/29; 423/604; 423/622; 424/635; 424/641; 428/402; 428/364; 977/773; 977/896

(57) ABSTRACT

Nanoparticles, compositions comprising the nanoparticles, and methods for manufacture and uses thereof are provided. In at least one specific embodiment, the nanoparticle can include at least one isotopically enriched metal oxide. The isotopically enriched metal oxide can be copper or zinc.

### CROSS-REFERENCE TO RELATED APPLICATION

This application claims priority to U.S. Provisional Patent Application having Ser. No. 61/479,753, filed Apr. 27, 2011, which is incorporated by reference herein.

BACKGROUND

1. Field

Embodiments described herein generally relate to nanoparticles. More particularly, such embodiments relate to nanoparticles comprising at least one isotopically enriched metal oxide, compositions comprising the nanoparticles, and methods for manufacture and uses thereof.

2. Description of the Related Art

The characterization and toxicological evaluation of nanoparticles can be important in the field of nanotechnology. One aspect of toxicological evaluation of nanoparticles relates to the ability to determine the bioaccumulation of nanoparticles in an organism, which will then help in ascertaining the toxicological impact of the nanoparticle. The need to carefully track the uptake and disposition of nanoparticles in order to understand toxicity as a function of dose at the site of action has been recognized. The increased availability of anthropogenic nanoparticles (both in terms of compositional variability and quantity produced), coupled with the bioavailability of nanoparticles within an organism makes the task of evaluating the bioaccumulation of nanoparticles within organisms difficult. One problem encountered can be how to differentiate between the background concentration and a newly accumulated concentration. This problem can be accentuated for bioavailable elements such as copper and zinc in comparison with less bioavailable elements such as silver and gold.

It is becoming increasingly recognized that the environmental implications of nanotechnologies should be more fully understood before significant further technological development takes place. Among the factors that can be considered with regard to the environmental implications of using specific nanomaterials include the quantities that reach the environment, plus the fate, bioavailability, and/or toxicity of the nanomaterials. In addition to being associated with potential novel forms of toxicity unique to nanosized particles, metal bearing nanoparticles also cause concern because of the potential toxicity of their dissolved components.

Research on the environmental impact of engineered nanoparticles is still hampered by a lack of reliable tools to detect, visualize, and quantitatively trace particle movement and transfer in complex environmental and biological systems. Labeling of nanoparticles to make them distinctive and thus easily detectable can be one way to resolve this problem. Various approaches have been developed including radioactive labeling with gamma or beta emitters and the use of various types of dyes for fluorescence microscopy imaging. Each of these labeling strategies requires post-synthesis manipulation of the nanoparticles to introduce the label and modification of the surface properties of the particles in the case of fluorescent dyes. Additionally, the use of gamma-emitting radioisotope tracers is limited to specific elements and to licensed laboratories. Such issues mean that only a limited number of relevant studies have been undertaken and that radiolabeled particles could most likely never be used as tracers in real scenarios.

Zinc and copper are ubiquitous elements in soils and sediments with concentrations ranging from a few to a few hundred μg/g. Large quantities of zinc are also released to the environment due to industrial activities (e.g., application of sewage sludge on soils or atmospheric emissions) which adds to the natural background concentration. Although potentially toxic at high doses, zinc is an essential element to organisms and it is required for the functioning of the metabolism. The ubiquitous presence of zinc in the environment can limit or hinder the ability to study its bioaccumulation dynamics particularly when considering short time exposures under environmentally realistic concentrations.

Stable isotope tracing may prove valuable for monitoring nanoparticles in the field and addressing emerging research questions but neither has yet been demonstrated. There are several technical challenges in creating stable isotope labeled nanoparticles and tracing their fate in biota including: availability of suitable isotopically enriched precursors for the synthesis of the material; quantitative measurement of the newly accumulated metal and distinguishing that from background concentrations, particularly while working at low exposure concentrations with essential metals like zinc and copper; and detection of the tracer in animal tissue following short time exposures at environmentally realistic concentrations.

There is a need, therefore, for nanoparticles comprising at least one isotopically enriched metal oxide, compositions comprising the nanoparticles, and methods for manufacture and uses thereof that can satisfy one or more of these conditions and/or are suitable for determining in-vivo and/or in-vitro biodistribution as biological/environmental nanotoxicity tracers.

### SUMMARY

Nanoparticles, compositions comprising the nanoparticles, and methods for manufacture and uses thereof are provided. In at least one specific embodiment, the nanoparticle can include at least one isotopically enriched metal oxide. The isotopically enriched metal oxide can be copper or zinc. In at least one specific embodiment, the composition can be or include two or more nanoparticles. The two or more nanoparticles can each include at least one isotopically enriched metal oxide. The isotopically enriched metal oxide can include copper or zinc.

In at least one specific embodiment, the method for making the nanoparticle can include converting a precursor of the isotopically enriched metal oxide to the nanoparticle that includes the at least one isotopically enriched metal oxide. In at least one other specific embodiment, the method for making the nanoparticle can include forming a precursor of the nanoparticle. The precursor can be an isotopically enriched precursor. The precursor can be isotopically enriched with a metal. The metal can include copper or zinc. The method can also include converting the precursor to the nanoparticle.

In at least one specific embodiment, a method for determining an uptake and extent to which nanoparticles have been distributed within a biological material can include introducing a composition comprising more than one nanoparticle to a biological material. The nanoparticles can include an isotopically enriched metal oxide. The metal can include copper or zinc. The method can also include determining a distribution of the nanoparticles in the biological material.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1a depicts Scanning Electron Microscopy (SEM) and Transmission Electron Microscopy (TEM) images of ⁶⁷ZnO samples obtained by hydrolysis of synthesized isotopically modified precursor (colloid) and its thermal decomposition product (powder).

FIG. 1b is a graphical depiction of X-ray diffraction (XRD) patterns of the two samples depicted to in FIG. 1a.

FIG. 1c is a graphical depiction of Dynamic Light Scattering (DLS) size distribution data of the samples in de-ionized water (DIW) and also shows a 50 nm polystyrene particle size standard (Duke Scientific) and a commercial sample of ZnO nanoparticles (Sigma Aldrich) for comparison.

FIG. 2 is a graphical depiction of zinc concentrations in snails exposed to dietborne ⁶⁷Zn for about 3 to 4 hours. The thin dark line across the exposure concentrations displays the mean background zinc concentration (total Zn) measured in 140 snails. The shaded areas represent the error surrounding the background zinc concentration; 1× and 3× the standard deviation (SD) of the mean background zinc concentration. The open symbols represent the detectable ⁶⁷Zn (newly accumulated Zn) after each exposure i.e., diatoms labeled with ⁶⁷Zn (triangles) and diatoms mixed with ⁶⁷ZnO nanoparticles (circles). Each concentration was derived from the total measured ⁶⁷Zn concentration minus background. The closed symbols represent the sum of the detectable zinc and the corresponding background concentrations, i.e., diatoms labeled with ⁶⁷Zn (triangles) and diatoms mixed with ⁶⁷ZnO nanoparticles (circles).

FIG. 3 provides XRD patterns of synthesized CuO nanoparticles and stable enriched ⁶⁵CuO nanoparticles.

FIG. 4 shows TEM images of (a) ⁶⁵CuO spherical nanoparticles, (b) CuO spherical nanoparticles, (c) ⁶⁵CuO nanorods, (d) CuO nanorods, (e) CuO spindles, and (f) a diagram indicating the dimensions of a spindle.

FIG. 5a depicts atomic force microscopy (AFM) images of CuO spherical nanoparticles and FIG. 5b depicts AFM images of CuO nanorods.

FIG. 6a is a graphical depiction of dissolved Cu²⁺ released from copper oxide nanoparticles in about 0.001 M NaNO₃ at about 25° C. for CuO and ⁶⁵CuO spherical nanoparticles. The data was fitted with a first order exponential growth equation.

FIG. 6b is a graphical depiction of dissolved Cu²⁺ released from copper oxide nanoparticles in about 0.001 M NaNO₃ at about 25° C. for CuO and ⁶⁵CuO nanorods. The data was fitted with a first order exponential growth equation.

FIG. 7 is a graphical depiction showing the release of dissolved copper expressed in proportion to an original concentration of nanoparticles compared to ionic copper used for control in the dissolution experiment. The data inset is a graphical depiction showing the release of ionic copper from copper oxide nanoparticles. All data was fitted with a first order exponential growth equation.

FIG. 8 is a graphical description of copper concentrations in snails exposed for 24 hrs to ⁶⁵CuO spherical nanoparticles dispersed in synthetic freshwater water. The thin dark line across the exposure concentrations displays the mean background Cu concentration (total Cu) measured in 200 snails. The shaded areas represent the error surrounding the averaged Cu concentration; 1× and 3× the SD of the mean. The open symbols represent the detectable ⁶⁵Cu (newly accumulated) after exposure to spherical isotopically modified ⁶⁵CuO spherical nanoparticles. Each concentration was derived from the total measured ⁶⁵Cu concentrations minus background. The closed symbols represent the sum of the detectable ⁶⁵Cu and the background Cu concentrations.

### DETAILED DESCRIPTION

One or more nanoparticles that can include at least one isotopically enriched metal oxide can be produced. The isotopically enriched metal oxides can include any one or more of the known isotopes of zinc and/or copper. The isotopical enrichment or “labeling” can be provided by a non-radioactive isotope of copper or zinc. For example, the isotopically enriched metal oxides can include, but are not limited to, ⁶⁴Zn, ⁶⁶Zn, ⁶⁷ZnO, ⁶⁸Zn, ⁷⁰Zn, ⁶³Cu, and ⁶⁵CuO. These zinc and copper isotopes are non-radioactive and stable. In another example, the isotopically enriched metal oxide can be or include ⁶⁷ZnO, ⁶⁵CuO, or a combination thereof. As used herein, the term “nanoparticles” can be abbreviated to “NPs” and the term “nanoparticulate” can be abbreviated to “nano”.

The nanoparticle can include the at least one isotopically enriched metal oxide. The nanoparticle can consist essentially of the at least one isotopically enriched metal oxide. The nanoparticle can consist of the at least one isotopically enriched metal oxide. As such, the nanoparticle can comprise, consist of, or consist essentially of ⁶⁴ZnO, ⁶⁶ZnO, ⁶⁷ZnO ⁶⁸ZnO, ⁷⁰ZnO, ⁶³CuO, and/or ⁶⁵CuO.

Nanoparticles are often taken to mean particles possessing a size of about 100 nm and less. As used herein, however, the term “nanoparticle,” refers to particles possessing an average particle size ranging anywhere from about 1 nm to about 1,000 nm or less than 1,000 nm. For example, the nanoparticles can have an average particle size ranging from about 1 nm to about 100 nm. In another example, the nanoparticles can have an average particle size ranging from a low of about 1 nm, about 10 nm, about 20 nm, about 30 nm, or about 40 nm to a high of about 75 nm, about 100 nm, about 150 nm, about 200 nm, about 300 nm, about 400 nm, or about 500 nm. In another example, the nanoparticles can have an average particle size ranging from a low of about 105 nm, about 120 nm, about 140 nm, about 160 nm, or about 180 nm to a high of about 250 nm, about 350 nm, about 450 nm, about 550 nm, about 650 nm, about 750 nm, or about 850 nm. The average particle size can be measured or estimated using a range of analytical methods. However, unless otherwise stated, the particle size is measured using transmission electron microscopy (TEM) using a Hitachi 7100 operating at an accelerating voltage of 100 kV.

Accordingly, nanoparticles discussed and described herein can have an average particle size ranging from about 1 nm to about 1,000 nm (for example, from about 1 nm to about 100 nm or, for example, from about 100 nm to about 1,000 nm) and can comprise, or consist of, or consist essentially of at least one isotopically enriched metal oxide, where the metal can be or include copper or zinc. Compositions comprising more than one of the nanoparticles are also discussed and described herein.

The nanoparticles can have a variety of shapes which can depend, at least in part, on the process used to make the nanoparticles. The shapes of the nanoparticles can include, but are not limited to, spheres, rods, and spindles. A cross-sectional geometry of the nanoparticles can be one or more of circular, ellipsoidal, triangular, rectangular, or polygonal, for example.

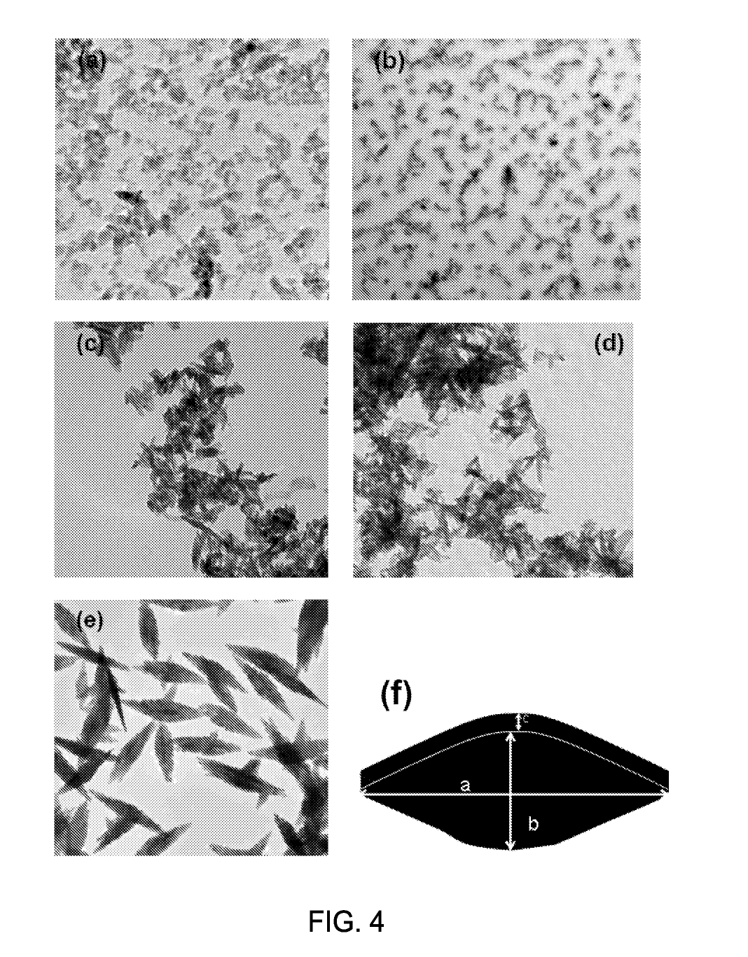

For embodiments that relate to spherical particle shapes, the average particle diameter can be about 1 nm to about 1,000 nm or about 1 nm to about 100 nm. For embodiments that relate to rod shaped nanoparticles (nanorods) the average length can be about 1 nm (for example at least about 5 nm) to about 1,000 nm or about 1 nm (for example at least about 5 nm) to about 100 nm. The width (or diameter) the rod shaped nanoparticles can also be about 1 nm to about 1,000 nm or about 1 nm to about 100 nm. The length to width ratio of rod shaped nanoparticles can be about 2:1 to about 10:1 or about 2:1 to about 20:1. For embodiments that relate to spindle shaped nanoparticles, for example CuO spindles; the spindle shaped nanoparticles can be envisaged as stretched diamond shaped particles (see, e.g., FIG. 4(f)). The average major axial width/diameter (b) of the spindle shaped particles can range from about 80 nm to about 1,000 nm. The average height/thickness (c) of the spindle shaped particles can range from about 10 nm to about 30 nm. The length (a) to width (b) ratio (or aspect ratio) can typically range from about 3:1 to about 7:1.

An enriched non-radioactive traceable metal isotope refers to a non-radioactive traceable isotope present at an abundance or amount that is greater than its natural abundance or amount. For example, the enriched non-radioactive (stable) traceable metal isotope can refer to a non-radioactive traceable isotope present at an abundance or amount that is far greater than its natural abundance or amount. The abundance can be characterized by its enrichment value. Non-radioactive traceable metal isotope enrichment refers to the relative percentage of the number of the non-radioactive traceable metal isotopes present in the nanoparticles compared to the total number of metal isotopes present in the nanoparticles. By way of example, a nanoparticle comprising an enriched ⁶⁵Cu isotope can include ⁶⁵Cu in an abundance which is equal to or greater than about 90%, greater than about 91%, greater than about 92%, greater than about 93%, greater than about 94%, or greater than about 95%. In another example, the metal isotope enrichment can be about 90% to about 99%, about 91% to about 98%, about 92% to about 99%, about 93% to about 97%, about 94% to about 98%, about 95% to about 99%, about 96% to about 99%, or about 91% to about 99% for ⁶⁵Cu. In another example, a nanoparticle comprising an enriched ⁶⁷Zn isotope can include ⁶⁷Zn in an abundance which is equal to or greater than about 70%, greater than about 75%, greater than about 80%, greater than about 85%, or greater than about 90%. In another example, the metal isotope enrichment can be from about 70% to about 95%, about 75% to about 90%, about 75% to about 95%, about 80% to about 95%, about 85% to about 95%, or about 80% to about 90% for ⁶⁷Zn.

The nanoparticles can be in the form of a powder. The nanoparticles can be dissolved in a liquid. The nanoparticles can be suspended in a liquid. For nanoparticles suspended in a liquid, the suspension can be a colloidal suspension.

As discussed and described herein, novel tracer materials using non-toxic starting materials that can be made using aqueous or non aqueous based synthesis are provided. The size and shape of the isotopically enriched metal oxide(s) can be varied or tuned. For example, different sizes and different shapes can be produced or obtained. In on example, different sizes of ⁶⁷ZnO nanoparticles and different shapes of ⁶⁵CuO nanoparticles can be produced or obtained. More generally, discussed and described herein are means for controlling the reactivity and/or behavior of nanoparticles in different media by tuning the shape of the nanoparticles.

Low cost approaches for making isotopically enriched nanoparticles using non-radioactive isotopes of zinc and copper have been developed. The starting materials for the synthesis of isotopically enriched nanoparticles of CuO and ZnO can be in an isotopically enriched or labeled form. Isotopically enriched zinc can be acquired as the metal or metal oxide (and in bulk/micron sized form). For example, isotopically modified zinc metal powder, e.g., ⁶⁷Zn, can be used as a starting material. The metal powder can be converted to a suitable precursor. A suitable precursor can be or include zinc acetate. Isotopically modified zinc powder can be heated with acetic acid to provide an isotopically modified zinc acetate precursor. The precursor can then be transformed into isotopically modified ZnO nanoparticles. Transforming the isotopically modified ZnO nanoparticles can be carried out via thermal decomposition and/or hydrolysis. For example, isotopically modified ZnO nanoparticles can be transformed via forced hydrolysis at elevated temperature (wet synthesis).

Thermal decomposition can be carried out in air at an elevated temperature. Thermal decomposition can take place at a temperature ranging from a low of about 150° C., about 200° C., or about 300° C. to a high of about 400° C., about 600° C., or about 800° C. For example, thermal decomposition can occur at a temperature of about 150° C. to about 800° C., about 200° C. to about 500° C., or about 250° C. to about 350° C. During thermal decomposition, heat can be applied over a period of about 5 minutes to about 1 hour. For example, heat can be applied during thermal decomposition for a time ranging from a low of about 5 minutes, about 10 minutes, or about 15 minutes to a high of about 30 minutes, about 40 minutes, or about 50 minutes. In at least one example, the thermal decomposition can occur at a temperature of about 250° C. to about 350° C. and for a time period of about 5 to about 30 minutes. In at least one other example, the thermal decomposition can occur at a temperature of about 300° C. for a time of about 15 minutes. The resultant product can be washed, for example with de-ionized water. The resultant product can be dried. For example, the resultant product can be dried in an oven for about 12 h, about 24 h, about 36 h, about 48 h, about 60 h, about 72 h, or more at a temperature of about 50° C.

Hydrolysis can be carried out by dissolving the precursor, e.g., the zinc acetate precursor, in a suitable solvent. A suitable solvent can include, but is not limited to, diethylene glycol (DEG). Dissolution can occur at an elevated temperature, e.g., about 80° C., followed by hydrolysis at a temperature of about 170° C. for about 1 hour following the addition of de-ionized water to provide a molar ratio of water to zinc of about 10:1.

The isotopically enriched ZnO and/or CuO nanoparticles can be present in the form of a powder, e.g., a dry powder, or they can be present in a suspension, for example a liquid suspension or a colloidal suspension. The isotopically enriched ZnO or CuO nanoparticles can be present in one or more of the following shapes: spherical, spindles, and/or rods.

By using stable isotopically modified ⁶⁷ZnO nanoparticles, it has been found that the uptake of zinc nanoparticles can be measured at a particularly low dietborne exposure concentration, for example less than about 15 μg/g (uptake from food). By comparison, for non-modified ZnO nanoparticles the distinction between exposed and background concentration was only achieved at a concentration of about 5,000 μg/g.

A suitable precursor for the synthesis of isotopically enriched copper oxide nanoparticles can be copper chloride (CuCl₂). Copper chloride can be used to provide control over the shape of the resulting nanoparticles. Isotopically modified CuCl₂, e.g., ⁶⁵CuCl₂.2H₂O, can be dissolved in water and combined with acetic acid and heated. The solution can be heated up to a temperature of about 100° C. A base, such as sodium hydroxide, can be added to the heated solution. Addition of the base can result in the formation of a precipitate which can be separated and washed with de-ionized water to provide copper oxide nanoparticles. The copper oxide nanoparticles can be phase pure copper oxide nanoparticles.

Methods for determining the extent to which the nanoparticles, or a composition comprising nanoparticles, uptakes within an organism or biological material are also discussed and described herein. The nanoparticles can be introduced to the biological material by a variety of known methods. Non-limiting examples for introducing the nanoparticles to an organism or biological material include intravenous administration; oral administration; dermal application; direct injection into muscle, skin, the peritoneal cavity and/or other tissues or other bodily compartments; or any combination thereof. The distribution of the nanoparticles in the biological material can be determined. The accumulation or concentration of the nanoparticles in the biological material can be determined. The distribution and the accumulation or concentration of the nanoparticles in the biological material can be determined.

### EXAMPLES

In order to provide a better understanding of the foregoing discussion, the following non-limiting examples are offered. Although the examples may be directed to specific embodiments, they are not to be viewed as limiting the invention in any specific respect. All parts, proportions, and percentages are by weight unless otherwise indicated.

### Particle Characterization

An X-ray diffraction (XRD), Enraf-Nonius diffractometer coupled to an INEL curved position-sensitive detector 120° (CPS 120 detector) with Cu K_{α} radiation was used for phase identification with STOE software. A high temperature XRD, Enraf-Nonius FR590 coupled with an INEL curved position-sensitive detector within a static beam-sample geometry, fitted with a GeniX system with Xenocs FOX2D CU 10_{—}30P mirror to generate ultra-high brightness Cu K_{α} was used to study the decomposition of a zinc acetate precursor into ZnO. A small portion of the precursor (few mg) was heated on a Pt holder from about 50° C. to about 800° C. with 50° C. increment steps with a 60 second temperature ramp time and a 30 second holding time at selected test temperatures (100° C., 200° C., 300° C., etc.). The shape and size of the particles were analyzed using Scanning Electron Microscopy (SEM) (Phillips XL30) and Transmission Electron Microscopy (TEM) (Hitachi 7100) operating at an accelerating voltage of 5 kV and 100 kV, respectively. ImageJ software was used to measure particle size from the SEM images (particles counted manually). Particle size in solution (DIW, 25° C.) was analyzed using Dynamic Light Scattering (DLS) on a Malvern Zetasizer Nano instrument (Malvern Instruments Ltd, UK) equipped with a He—Ne 633 nm laser. Colloids were prepared for DLS measurement by diluting about 100 μL in about 10 ml of DIW. Powders were suspended in DIW (about 1 mg in about 10 ml) with the aid of an ultrasonic probe UP100H (100 W, 30 kHz, 30 seconds). The surface charge of the samples were measured in DIW at about 25° C., pH=7.4, using the zeta potential module of the Malvern instrument.

### Example 1

### Synthesis of ZnO and Isotopically Enriched ZnO Nanoparticles (⁶⁷ZnO-NP)

ZnO nanoparticles were synthesized from non-isotopically modified zinc metal powder (AnalaR, BDH Chemicals, CAS 7740-66-6) to optimize the synthesis conditions (reaction temperature, time, and precursor concentration). The optimized protocol was then followed while working with isotopically enriched metal powder (the enrichment level of ⁶⁷Zn used was 89.6% and was purchased from Isoflex, Moscow, Russia).

The metal powder (500 mg) was heated with acetic acid (100%, 50 ml, AnalaR, BDH Laboratory Supplies) at about 80° C. for 3 days, which yielded approximately 1 g of a zinc acetate precursor (ZAP). After about 48 hours of drying at about 50° C., the ZAP was transformed into ZnO nanoparticles via either thermal decomposition or forced hydrolysis in diethylene glycol (DEG, ReagentPlus 99%, Sigma Aldrich) using a protocol modified from Feldmann, C., Jungk, H. O., 2001: Polyol mediated synthesis of nanoscale oxide particles; Angewandte Chemie International Edition 40(2), 359-362, which is discussed and described in more detail below.

Thermal Decomposition: a plurality of zinc acetate precursor samples were decomposed in air in porcelain crucibles that were placed in a preheated Griffin electric furnace. Following decomposition, the samples were cooled to room temperature in a desiccator. Both the effect of changing the temperature (150° C. to 800° C.) and the time (5 minutes to 56 hours) had on the transformation of the zinc acetate precursor into the final product (ZnO) and on the particle size of the final product were tested. An isotopically modified particle sample was also prepared by thermal decomposition (in air) at about 300° C. for about 15 min. The prepared sample was washed three times with deionized water (DIW) and dried in an oven at about 50° C. for about 24 hours. The reaction yield from the thermal decomposition synthesis route was about 25%. From approximately 200 mg of the precursor, approximately 25 mg of the final product ZnO was obtained via this route.

Hydrolysis (wet synthesis): a plurality of zinc acetate precursor samples were dissolved in diethylene glycol (DEG) at a temperature of about 80° C. The dissolved precursors were hydrolyzed (in an oil bath) for about 1 hour at a temperature of about 170° C. following addition of DIW (Millipore, <18 MΩ·cm) to provide a molar ratio of water to zinc of 10:1. The following concentrations of the precursor were tested: about 500 mg, about 300 mg, and about 100 mg in about 50 ml of DEG. Isotopically modified particles were prepared using about 100 mg of the precursor hydrolyzed with about 100 μL of DIW for about 1 h in DEG at a temperature of about 170° C.

A high temperature XRD stage was used to study the transformation and changes in crystallinity and particle size of the precursor as a function of temperature. Zinc oxide peaks were observed to emerge on the pattern as the temperature reached about 250° C. to about 300° C. indicating the onset of the precursor transformation. As the temperature increased, ZnO peaks became sharper indicating an increase in crystallinity as well as in the crystallite size of the ZnO.

To decide on the optimum temperature for thermal decomposition, the particle size distribution in samples heated at selected temperatures were measured using Dynamic Light Scattering (DLS). At a temperature of about 150° C. and about 800° C., DLS indicated the formation of large sedimenting particles. The sample heated to a temperature of about 600° C. showed a bimodal distribution with peaks at about 130 nm and about 720 nm whilst the sample heated at 300° C. appeared to be optimum, showing a monomodal particle size distribution with a peak at about 190 nm.

Based on the XRD data and on the particle size measurements (SEM and DLS) of the powders decomposed at varying temperatures, a temperature of about 300° C. was selected as an optimal decomposition temperature to obtain pure ZnO with the smallest and most uniform particle size distribution (about 200 nm to about 300 nm average agglomerate size from DLS).

The thermal decomposition route to produce ZnO produced agglomerated particles before dispersing in any media and they were also relatively polydispersed in both size and shape. The hydrolysis (wet synthesis) route lead to monodispersed samples and offered better possibilities of tuning the size and/or shape within one synthesis protocol. Forced hydrolysis in DEG proved to be an effective protocol, and repeatedly provided pure phase monodispersed ZnO colloidal suspensions. By experimenting with the synthesized zinc acetate precursor the size of ZnO obtained via this method was tuned in the range from about 150 nm to about 550 nm by changing the precursor concentration from about 100 mg to about 500 mg in about 50 ml. The smallest particles were obtained at the lowest precursor concentration and for isotopic modification only this precursor concentration was chosen and one size obtained.

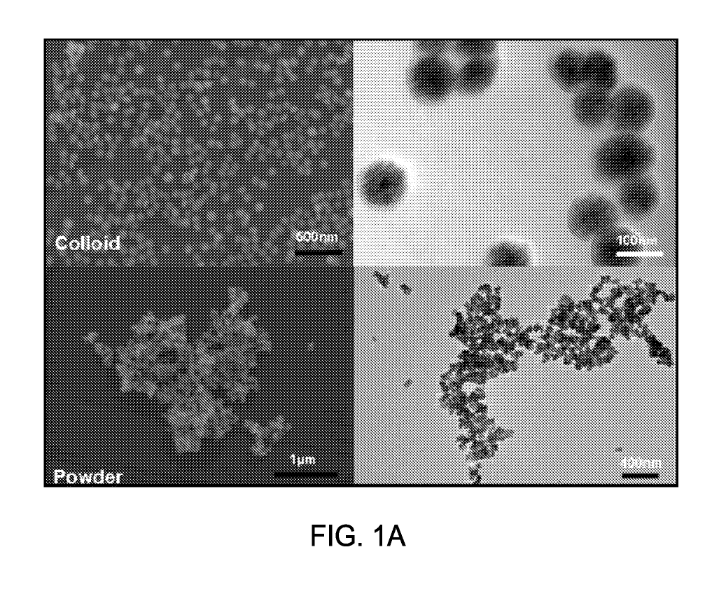

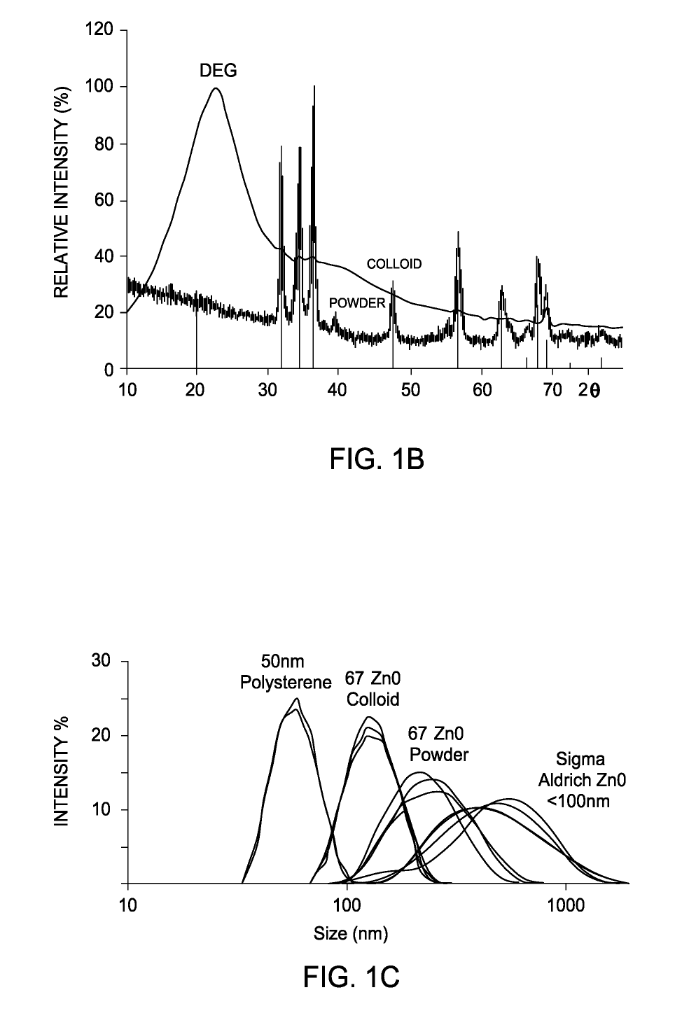

Having optimized the synthesis conditions, the above experiments were repeated with isotopically modified zinc metal as a starting material and two sets of isotopically modified ZnO particles were produced in a powder form following thermal decomposition of the precursor at a temperature of about 300° C. for about 15 minutes and in a colloidal suspension form following forced hydrolysis in DEG (about 100 mg of precursor in about 50 ml DEG). The characterization data for both samples are shown in FIG. 1a (SEM and TEM), FIG. 1b (XRD), and FIG. 1c (DLS).

The XRD patterns in FIG. 1b confirmed the presence of pure phase ZnO in both the powder and colloid samples. In the latter, a large scattering peak was observed at approximately 2θ=22.4, which can be attributed to the polymer in which the particles were suspended as the same peak was observed on the pattern from the polymer sample only and the intensity of this peak was observed to decrease as the colloid sample was pre-concentrated by washing and thus polymer was removed.

Sample 1 (powder) was agglomerated with individual particles varying in size from about 20 nm to about 70 nm (TEM) and also of variable shape (i.e., spherical, trigonal, rods). The average size of agglomerates, as indicated by DLS measurements, was about 245 nm in this sample. Sample 2 (colloid) was monodispersed in size and shape with highly symmetrical particles of average size 125 nm (from DLS measurements) and about 110 nm±11 nm; n=100, (from SEM). The surface charge was measured for both samples and was negative (−17.7±0.64 mV) for Sample 1 (powder) and positive (23.6±5.17 mV) for Sample 2 (colloid). Without wishing to be bound by theory, it is believed that the difference in surface charge between the powder and colloid samples was likely due to changes in the surface properties of the powder (surface speciation) as a result of the process of drying and dehydration.

The stability of the colloidal suspension (Sample 2) was followed for 4 months by taking DLS measurements over time. There was no indication of significant particle growth or agglomeration over time. Changes over time are an important consideration because environmentally realistic eco-toxicological tests often necessitate exposures for long periods (e.g., weeks) and/or storage of stocks before use.

### Dietborne Exposures to Enriched ⁶⁷Zn

Freshwater snails (Lymnaea stagnalis) were reared in a laboratory in moderately hard water (MOD, hardness of about 80 mg to about 100 mg of CaCO₃ L⁻¹; pH of about 7.0, U.S. EPA, 2002). Three days prior to each experiment, snails of a restricted size range (mean soft tissue dry weight of about 6.8+0.6 mg 95% CI, n=112) were transferred to a 10 L glass aquarium filled with MOD water. Food was withheld during this period.

The benthic diatom Nitzschia palea was grown axenically for several generations in an S-diatom medium (Irving, E. C., Baird, D. J., Culp, J. M., 2003: Ecotoxicological responses of the mayfly Baetis tricaudatus to dietary and waterborne cadmium: Implications for toxicity testing; Environmental Toxicology and Chemistry 22, 1058-1064). Diatoms were harvested onto a 1.2 μm Isopore membrane filter (Millipore) and rinsed with synthetic soft water (SO, hardness of about 40 mg to about 48 mg of CaCO₃ L⁻¹; pH of about 7.0, U.S. EPA, 2002).

To achieve a dietborne zinc concentration of up to about 1,000 μg g⁻¹, the protocol described by Croteau, M-N., Luoma, S. N., 2009: Predicting dietborne metal toxicity from metal influxes; Environmental Science and Technology 43, 4915-4921, was used which involved labeling algae with enriched stable metal isotopes. Dietborne zinc concentrations achieved using this procedure were limited by the algae tolerance to the acute dissolved metal exposure. Algae were re-suspended into a 20 mL acid-washed glass scintillation vial filled with SO water spiked with different concentrations (from about 10 μg L⁻¹ to about 5,000 μg L⁻¹) of a commercially purchased standard which was isotopically enriched in ⁶⁷Zn (94%, Trace Sciences International, Canada) for about 24 h. After exposure, labeled-diatoms were harvested onto a 1.2 μm Isopore™ membrane filter (Millipore) and rinsed with SO water. Small sections of the filters (less than about 10% of the filter area, which is approximately 17 cm²) that held the labeled diatoms were sampled and dried for about 24 hours at about 40° C. prior to metal analysis. The remaining filters coated with diatoms labeled with enriched ⁶⁷Zn were offered as food to L. stagnalis.

To achieve dietborne zinc concentrations higher than about 1000 μg g⁻¹, the protocol described by Croteau, M-N., Dybowska, A., Luoma, S. N., Valsami-Jones, E., 2010: A novel approach reveals that ZnO nanoparticles are bioavailable and toxic after dietary exposures; Nanotoxicology, In Press, doi: 10.3109/17435390.2010.501914, was used which involved amending diatom mats with isotopically modified nanoparticles. The use of metal enriched nanoparticles allowed a higher concentration to be achieved as compared to if algae were exposed to dissolved metal.

Serially diluted suspensions of isotopically modified ZnO nanoparticles (made by forced hydrolysis (average size about 110 nm±11 nm, n=100, surface charge of about 24 mV±5 mV) were poured onto algal mats and filtered therethrough under low vacuum (<10 mm Hg) to deposit particles. Small sections of the filters (less than about 10% of the total filter area) that held the diatoms amended with ZnO nanoparticles were sampled and dried for about 24 hours at about 40° C. prior to metal analysis. The remaining filters coated with diatoms mixed with isotopically enriched ⁶⁷ZnO NPs were offered as food to L. stagnalis.

### Dietborne Uptake Experiments

In connection with the dietborne uptake experiments, for each treatment, 8-10 acclimated snails were transferred to a feeding chamber made from a 150 ml polypropylene vial that had two 4 cm diameter holes (opposite edges) covered with about 100 μm nylon mesh. Each feeding chamber was acid-washed prior to being partially submerged in a 20 L glass tank filled with MOD water as described in Croteau, M-N., Dybowska, A., Luoma, S. N., Valsami-Jones, E., 2010: A novel approach reveals that ZnO nanoparticles are bioavailable and toxic after dietary exposures; Nanotoxicology, In Press, doi: 10.3109/17435390.2010.501914. For each treatment, a filter holding the ⁶⁷Zn labeled diatoms was laid flat on the bottom of the feeding chamber. Snails were exposed to the labeled food for about 3 to 4 hours. After feeding on the labeled food, the snails were removed, rinsed with DIW and allowed to depurate for about 48 hours in the 20 L glass tanks filled with MOD water. Unlabeled lettuce was provided ad libitum during depuration. After depuration, the snails were frozen.

### Sample Preparation, Analysis and Calculation of Accumulated ⁶⁷Zn Concentrations

Partially thawed L. stagnalis were dissected to remove soft tissue, placed individually on a piece of acid-washed TEFLON® sheeting and allowed to dry at about 50° C. for 3 days. Dried snails and diatoms were weighed and digested at room temperature in TEFLON® vials with concentrated nitric acid (Baker Ultrex II grade, 100 μL mg dry wt sample⁻¹) for 5-7 days. Hydrogen peroxide (Baker Ultrex II grade, 40 μL mg dry wt sample⁻¹) was added prior to final dilution with DIW water. Similar weight samples of the certified reference material NIST-2976 (mussel tissue from National Institute of Standards and Technology) were also submitted to the same digestion procedure during each analytical run.

Water and digested samples were analyzed for the naturally occurring stable isotopes of zinc (⁶⁴Zn, ⁶⁶Zn, ⁶⁷Zn, ⁶⁸Zn and ⁷⁰Zn) by Inductively Coupled Plasma-Mass Spectrometry (ICP-MS) as described in Croteau, M-N., Luoma, S. N., Topping, B. R., Lopez, C. B., 2004: Stable metal isotopes reveal copper accumulation and loss dynamics in the freshwater bivalve Corbicula; Environmental Science and Technology 38, 5002-5009. To account for instrument drift and change in sensitivity, internal standardization was performed by the addition of germanium (⁷⁴Ge) to all samples and standards, apart from the calibration blanks. One of the standards was re-analyzed after every 10 samples. Deviations from the standard value were in general less than about 5% for the analyzed isotopes.

An isotope tracing technique that allows tracking newly accumulated tracers independent from background levels was used, Croteau, M-N., Luoma, S. N., Topping, B. R., Lopez, C. B., 2004: Stable metal isotopes reveal copper accumulation and loss dynamics in the freshwater bivalve Corbicula; Environmental Science and Technology 38, 5002-5009. The relative abundance of ⁶⁷Zn tracer (i.e., p⁶⁷) was determined using the signal intensities of each isotope in the calibration standards:

$${p}^{67} = {Intensity ( \frac{{}^{67} Zn}{{}^{64} Zn + {}^{66} Zn + {}^{67} Zn + {}^{68} Zn + {}^{70} Zn} )}_{standard} \qquad ( 1 )$$

Concentrations of tracer in the experimental organisms ([⁶⁷Zn]_{ê}) were calculated as the product of p⁶⁷ and the total metal concentrations inferred by the ICP-MS software from tracer intensity ([T⁶⁷Zn]):

[⁶⁷Zn]_{ê}=p⁶⁷×[T⁶⁷Zn] (2)

The original load of tracer)([⁶⁷Zn]_{ê}⁰) that occurred in each sample in the absence of a spike was calculated as the product of p⁶⁷ and the total metal concentrations inferred from the intensity of the most abundant isotope that was minimally affected by isobaric and polyatomic interferences, Croteau, M-N., Luoma, S. N., Pellet, B., 2007: Determining metal assimilation efficiency in aquatic invertebrates using enriched stable metal isotope tracers; Aquatic Toxicology 83, 116-125, (e.g., ⁶⁶Zn):

[⁶⁷Zn]_{ê}⁰=p⁶⁷×[T⁶⁶Zn] (3)

The net tracer uptake (Δ[⁶⁷Zn]_{ê}) was derived from the total experimental metal concentration ([⁶⁷Zn]_{ê}, equation 2) minus the pre-existing concentration of tracer ([⁶⁷Zn]_{ê}⁰, equation 3):

Δ[⁶⁷Zn]_{ê}=p⁶⁷×([T⁶⁷Zn]−[T⁶⁶Zn]) (4)

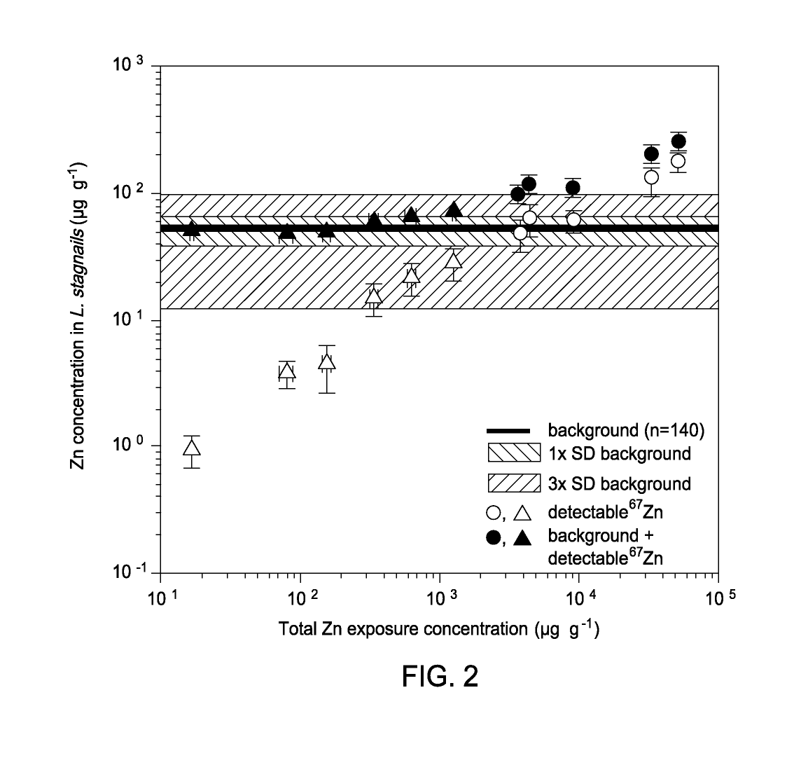

Experiments were conducted to determine whether ⁶⁷Zn enriched nanoparticles could be detected in animal tissues after short dietary exposures. To do this, freshwater snails were fed (L. stagnalis) diatoms mixed with different amounts of the synthesized ⁶⁷ZnO nanoparticles (made by forced hydrolysis) and then fed unlabelled food to clear their gut. The ⁶⁷Zn retained after gut clearance (about 48 hours as defined by Croteau, M-N., Luoma, S. N., Pellet, B., 2007: Determining metal assimilation efficiency in aquatic invertebrates using enriched stable metal isotope tracers; Aquatic Toxicology 83, 116-125) was used to define assimilation. Snails were fed diatoms labeled with ⁶⁷Zn to show how a tracer can improve experimental sensitivity, thereby overcoming the limitations imposed by the high natural concentrations of total zinc in animals and the environment. FIG. 2 shows that zinc concentration as low as about 1 μg g⁻¹ can be detected in freshwater snails exposed to about 15 μg g⁻¹ of dietborne zinc when an enriched stable isotope of zinc was used as a tracer (⁶⁷Zn). In contrast, exposures about 333 times higher were required to detect zinc uptake if no tracer was used. That is, detection is significant at a zinc exposure concentration only above about 5,000 μg g⁻¹ if no tracer is used. Thus, the enriched stable isotope technique is sufficiently sensitive to determine the uptake rate of zinc in L. stagnalis at an exposure equivalent to the lowest concentrations of zinc that might be expected in environmental media. Without the tracer, uptake rates can only be determined at concentrations equivalent to some of the most contaminated zinc conditions (or much longer exposure times), reducing the feasibility of studying a range of environmentally realistic exposure conditions.

### Example 2

### Synthesis of CuO & Isotopically Enriched ⁶⁵CuO Nanoparticles (⁶⁵CuO-NP)

Two types of copper oxide (non-isotopically modified CuO and isotopically modified ⁶⁵CuO) nanoparticles with different shapes (spheres, rods, and spindles) were synthesized using wet chemistry. About 0.02 M of CuCl₂.2H₂O was dissolved in water and about 500 μl of glacial acetic acid was added to the solution. The solution was then heated to about 100° C. followed by rapid addition of about 0.8 g NaOH at a temperature of about 85° C. This resulted in formation of a black precipitate, which was centrifuged out and repeatedly washed with de-ionized water to obtain phase pure copper oxide nanoparticles. The same synthesis route was also used to prepare ⁶⁵CuO nanoparticles. Isotopically modified CuCl₂.2H₂O with about 99% enrichment was purchased form Trace sciences, USA. The synthesized nanoparticles were washed three times in de-ionized water prior to further characterization to remove any un-reacted regents or by-products of the synthesis reaction.

The synthesis method used to develop the CuO particles was adjusted to achieve specific shapes of particles. For example, spherical shaped ⁶⁵CuO and CuO NPs were synthesized by dissolving about 0.51 g of CuCl₂.2H₂O in a round bottom two neck flask containing about 150 ml of de-ionized water and about 500 μl of glacial acetic acid. The solution was heated to a temperature of about 100° C. in a mantle stirrer, at which point about 0.8 g of NaOH pellets were added to observe a black precipitate of spherical copper oxide nanoparticles.

For rod shaped copper oxide NPs, about 0.51 g of CuCl₂.2H₂O was dissolved in a round bottom two neck flask containing about 150 ml of de-ionized water and heated to a temperature of about 100° C. in a mantle stirrer, at which point about 0.8 g of NaOH pellets was added to observe a black precipitate of rod-shaped copper oxide nanoparticles.

Spindle shaped CuO particles were prepared by dissolving about 0.51 g of CuCl₂.2H₂O at room temperature in a round bottom two neck flask containing about 150 ml of de-ionized water, about 500 μl of glacial acetic acid, and about 0.8 g of NaOH pellets. The solution was then heated to about 100° C. and the color of the solution gradually started to blacken. The solution on reaching a temperature of about 100° C. was kept at that temperature for about 10 minutes, after which the spindle shaped particles were suspended out and washed three times with water for further use.

A diluted suspension of the nanoparticles was deposited on a copper grid for TEM imaging (Hitachi 7100, 100 kV). The hydrodynamic size and zeta potential of the nanoparticles were measured using a Malvern Zetasizer (Malvern Instruments) at a concentration of about 800 mg 1⁻¹ at a pH of about 7 and a temperature of about 22° C. X-ray diffraction was performed on the copper oxide nanoparticles using an Enraf-Nonius diffractometer coupled to an INEL CPS 120 position-sensitive detector with Cu-K_{α} radiation and the phase identification was performed using STOE software. For AFM measurements, diluted CuO suspensions were deposited onto glass slides and allowed to air dry in a clean environment. AFM was conducted on the samples, using an Asylum MFP-3D-SA (Santa Barbara, USA) instrument in AC mode. The samples were scanned in air using an Olympus AC-240TS tip (spring constant 2 N m⁻¹). ICP-AES analysis was performed to determine the initial concentration of the copper oxide nanoparticles in aqueous suspension and also measure the solubility of the nanoparticles as described below.

### Dissolution Studies

About 10 mL of a CuO nanoparticle suspension in water were put inside a dialysis bag (MWCO=12,400 Da) and transferred into 250 mL plastic bottles (Nalgene) containing about 200 mL of about 0.001 M NaNO₃ (pH about 6.7). Appropriate blanks were also included in the experimental set up to control for a potential contamination from reagents and containers. All of the samples were thoroughly washed before the dissolution experiment. The starting CuO concentration inside the dialysis bag for all the dissolution experiments of the different shaped nanoparticles were kept constant at about 750 mg 1⁻¹. The bottles were incubated at a temperature of about 25° C. and about 200 rpm. All of the samples were triplicated and appropriate blanks were also included in the experimental set up. Aliquots of about 1 mL were taken from the media outside the dialysis bag at regular intervals, acidified with about 5% HNO₃ and the concentration of copper was then measured using inductively coupled plasma-atomic emission spectroscopy (ICP-AES, Varian Instruments). At the end of the dissolution experiment, suspensions inside the dialysis bag were examined under TEM to ascertain the presence of particles.

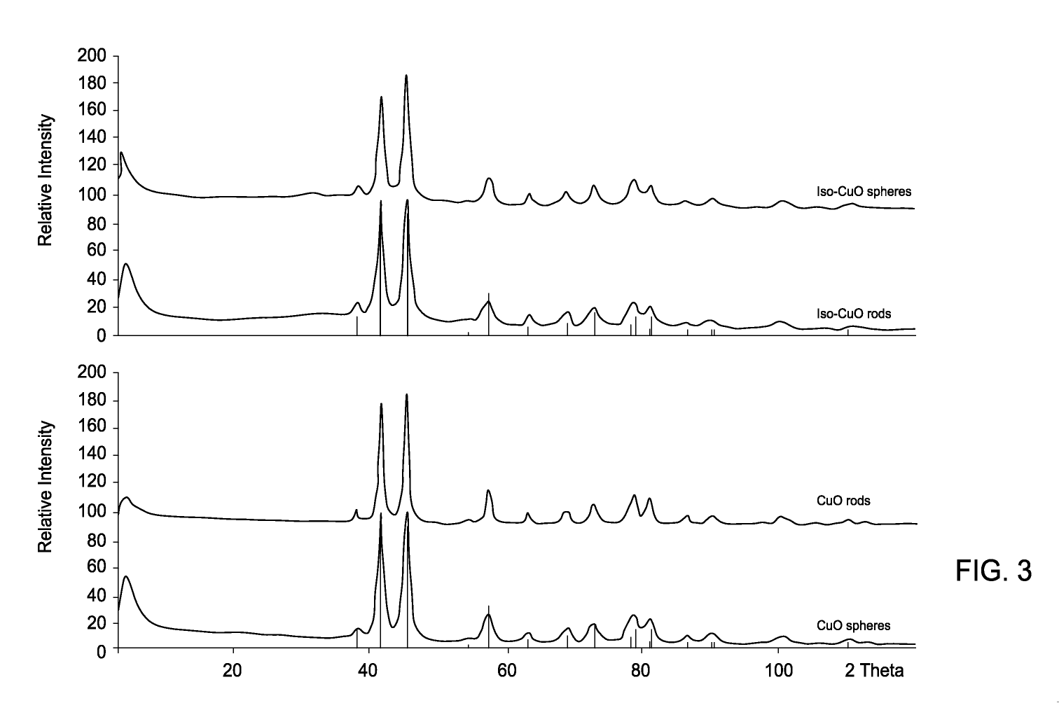

The size, shape and crystal structure of the synthesized copper oxide nanoparticles are shown in FIGS. 3, 4, and 5 along with the appropriate abbreviations of the samples shown in Table 1.

TABLE 1
characterization of the copper oxide nanoparticles

| | | | BET | | *DLS | Zeta potential (mV) | |
|---|---|---|---|---|---|---|---|
| Sample | Shape | Symbol | (m² g⁻¹) | TEM (nm) | (nm) | Stock | @750 ppm |
| CuO | sphere | CuO-s | 60 ± 1 | 7 | 76 ± 1 | +43 ± 1 | +43 ± 1 |
| ⁶⁵CuO | sphere | ⁶⁵CuO-s | -nd- | 7 | 82 ± 1 | +42 ± 1 | +42 ± 1 |
| CuO | rods | CuO-r | 51.5 ± 1.1 | 7(w) × 50(l) | -nd- | +36 ± 1 | +27 ± 1 |
| ⁶⁵CuO | rods | ⁶⁵CuO-r | -nd- | 7(w) × 40(l) | -nd- | +35 ± 1 | +24 ± 2 |

w—width,
l—length,
-nd- = not determined;
*DLS measurements conducted in water at a concentration of about 750 mg l⁻¹.

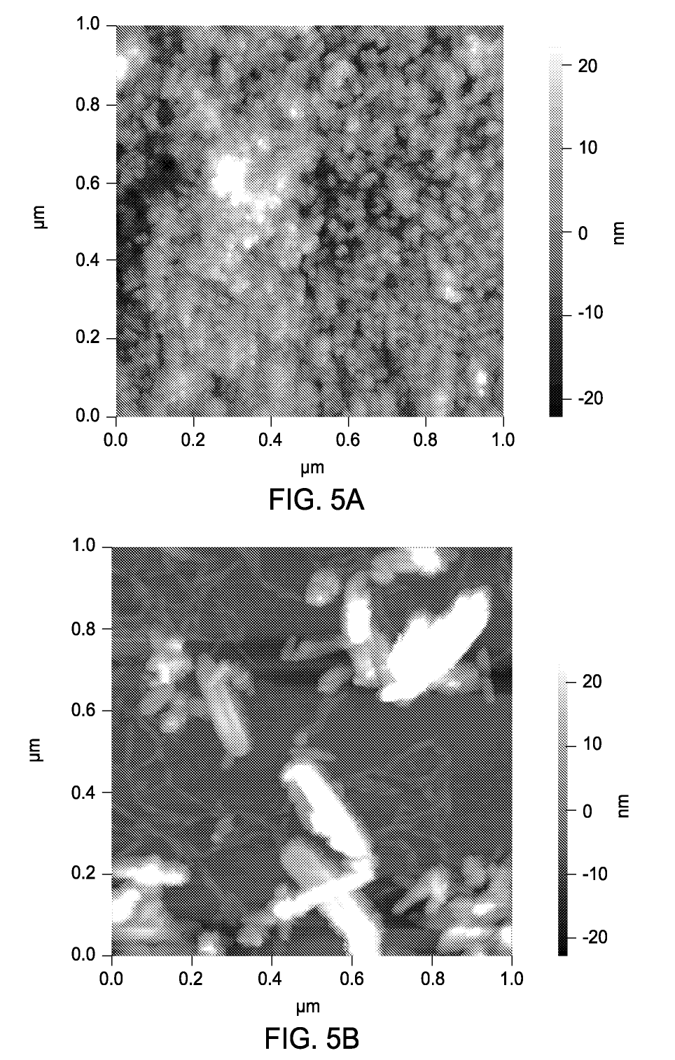

FIGS. 4(a) and (b) show TEM images for CuO-s and ⁶⁵CuO-s NPs, respectively and FIG. 5(a) shows the AFM image for CuO-s NPs. The spherical NPs for both types of copper oxide had a similar TEM size (about 7 nm) and appeared to be monodispersed and in a non-aggregated state. The spherical nature of the nanoparticles was also confirmed through AFM imaging, as the height of the particles was measured to be about 9 nm (FIG. 5(a)) using AFM. In the case of copper oxide nano rods, the particles were measured to be about 5 nm to about 7 nm in width and about 30 nm to about 50 nm in length using TEM. AFM measurement on the samples showed the height (equivalent to the diameter) of individual nanorods as about 7±1 nm (FIG. 5(b)). XRD was used to evaluate the phase of the nanoparticles and for both the shape (rods and spheres) and type (isotopic and non-isotopic) of nanoparticles the particles were of pure tenorite phase (ICDD 48-1548) as shown in FIG. 3. The specific surface areas (measured using BET) for the CuO-s and CuO-r NPs were about 60 m² g⁻¹ and about 51 m² g⁻¹, respectively. The increase in the specific surface area for the samples (spheres) was evident from the morphology of the particles, as shown in TEM. This increase in surface area for the spherical samples can play a role in the reactivity of the samples. The hydrodynamic size of CuO-s and ⁶⁵CuO-s NPs was measured as about 76 nm and about 82 nm, respectively using DLS. The stability of the nanoparticle suspension in water was measured using zeta potential measurements and the suspensions were found to be stable, as indicated by their high zeta values (Table 1).

The dissolution of nanoparticles was also measured in about 0.001 M NaNO₃ solution using dialysis membranes. Prior to the dissolution experiments, the zeta potential of the suspensions at a concentration of about 750 ppm showed that the nanoparticles remained as a stable suspension as indicated by the higher zeta values, as shown in Table 1. The dissolution experiments designed in this study ensured that the dissolved fraction of copper only contributed to the measurement and not the nanoparticles, by using a low molecular weight cut off value (MWCO=12,400 Da equivalent to 1 nm) dialysis membrane that will effectively only allow the passage of free dissolved/complexed ionic species. The aliquots for dissolved copper measurements were collected from outside the dialysis bag thus minimizing any risk of spillage of CuO NPs suspension from inside the bag to the outside solution sampled and also allowing data to be gathered for the entire duration of the study from the same individual sample. Bulk copper oxide particles and soluble copper nitrate compound were used as two controls to test the feasibility of the experiment.

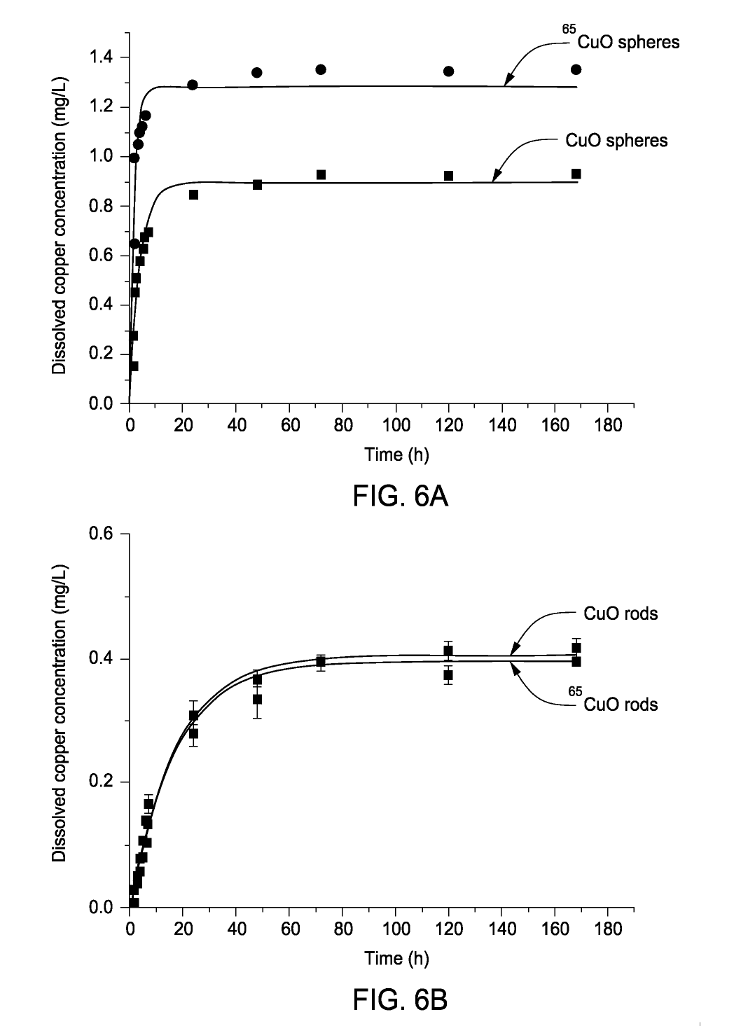

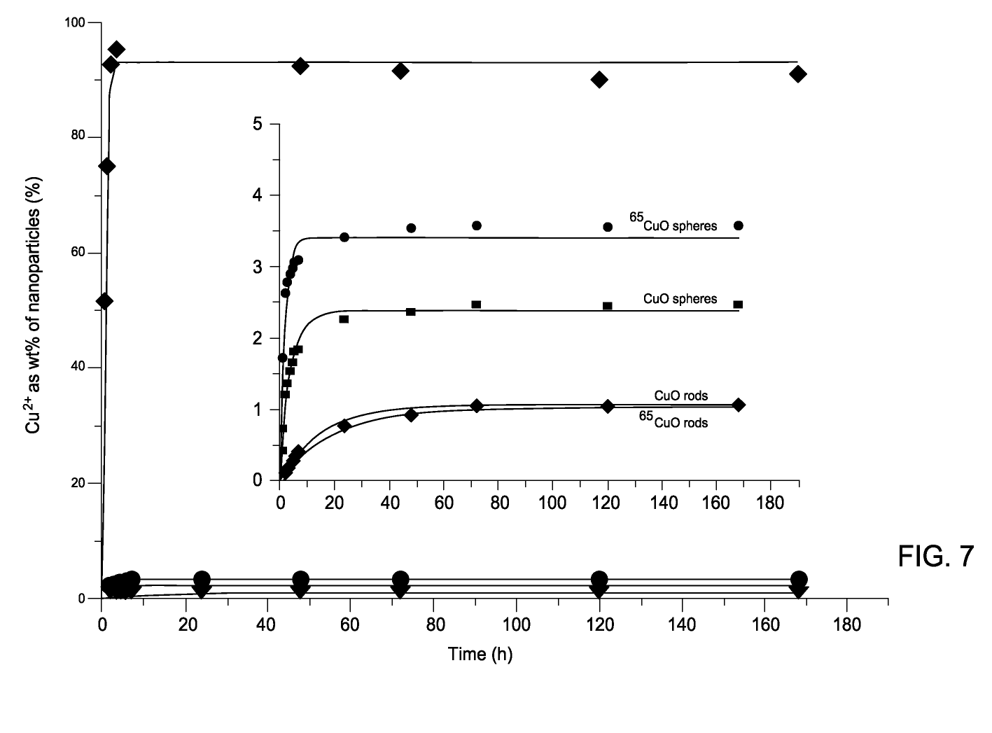

In FIGS. 6a and 6b the dissolved copper (Cu²⁺) fraction released from the synthesized CuO and ⁶⁵CuO nanoparticulate system for a period of up to 7 days is shown. From CuO-s and ⁶⁵CuO-s NPs up to about 1.3 ppm and about 0.9 ppm of dissolved copper was released. Whereas, in the case of CuO-r and ⁶⁵CuO-r NPs up to about 0.4 ppm of dissolved copper was released. In the case of CuO-s and ⁶⁵ CuO-s spherical nanoparticles (FIG. 6a), the equilibrium concentration was reached within about 24 hrs of the experiment and no significant change was observed for the rest of the duration. In the case of CuO-r and ⁶⁵CuO-r nanorods (FIG. 6b) the equilibrium concentration was reached after about 60 hours and remained unchanged for the rest of the period. The dissolution of the nanoparticles in terms of mass (%) is represented in FIG. 7, where it can be observed that diffusion of Cu²⁺ through the dialysis membrane was fast and undisrupted by interaction of Cu²⁺ ions with the membrane, as evident by up to about 95% recovery for CuNO₃. In the case of rod shaped nanoparticles, both types of nanoparticles showed a release of up to about 1% and for spherical nanoparticles the release was a maximum of about 3.5% for ⁶⁵CuO-s and about 2.4% for CuO-s NPs. In comparison, no dissolution from the micron sized CuO was observed for the same duration (data not shown). No significant differences in the size of the nanoparticles after dissolution from inside the bag were observed for both types apart from some agglomeration for CuO-s and ⁶⁵CuO-s NPs. The significant difference in the dissolution between the rod shaped and spherical nanoparticles could be attributed to the differences in the surface area arising from the different shapes of the NPs. Apart from influencing the equilibrium concentration, the shape of the nanoparticles also played an important role on the rate of dissolution. The release of Cu²⁺ was fastest from the ionic source, followed by the spherical shaped nanoparticles, and slowest for the rod shaped nanoparticles. The importance of this observation is in designing the nanotoxicological studies with these particles, where the dissolution of the NPs must be considered while conducting exposure studies.

### Waterborne Uptake Experiments

Acute waterborne exposures were conducted using the isotopically modified ⁶⁵CuO spherical NPs in moderately hard water (US EPA 2002). Snails (Lymnaea stagnalis) of a restricted size range (mean dry weight of 13.4±1.9 mg 95% CI, n=47) were exposed for 24 hrs to a range of Cu concentrations in acid-washed 1 L HDPE containers. After exposure snails were sacrificed, digested and analyzed as described by Croteau, M-N., Luoma, S. N., Topping, B. R., Lopez, C. B., 2004: Stable metal isotopes reveal copper accumulation and loss dynamics in the freshwater bivalve Corbicula; Environmental Science and Technology 38, 5002-5009. Before and after exposure, water samples were taken from each vial, acidified (Baker Ultrex II grade, 2% HNO₃ final concentrations) and analyzed by ICP-MS. An isotope tracing technique that allows tracking newly accumulated tracers, independently from background levels, was used. Briefly, the relative abundance of ⁶⁵Cu tracer, i.e., p⁶⁵, was determined using the signal intensities of each isotope in the calibration standards, i.e.,

$${p}^{65} = Intensity ( \frac{{}^{65} Cu}{{}^{65} Cu + {}^{63} Cu} ) \qquad ( 5 )$$

Concentrations of tracer in the experimental organisms ([⁶⁵Cu]_{ê}) were calculated as the product of p⁶⁵ and the total metal concentrations inferred by the ICP-MS software from tracer intensity ([T⁶⁵Cu]), i.e.

[⁶⁵Cu]_{ê}=p⁶⁵×[T⁶⁵Cu] (6)

The original load of tracer ([⁶⁵Cu]_{ê}⁰) that occurred in each sample in the absence of a spike was calculated as the product of p⁶⁵ and the total metal concentrations inferred from the intensity of the most abundant isotope ([T⁶³Cu]), i.e.

[⁶⁵Cu]_{ê}⁰=p⁶⁵×[T⁶³Cu] (7)

The net tracer uptake (Δ[⁶⁵Cu]_{ê}) was derived from the total experimental metal concentration ([⁶⁵Cu]_{ê}, equation 6) minus the pre-existing concentration of tracer ([⁶⁵Cu]_{ê}⁰, equation 7).

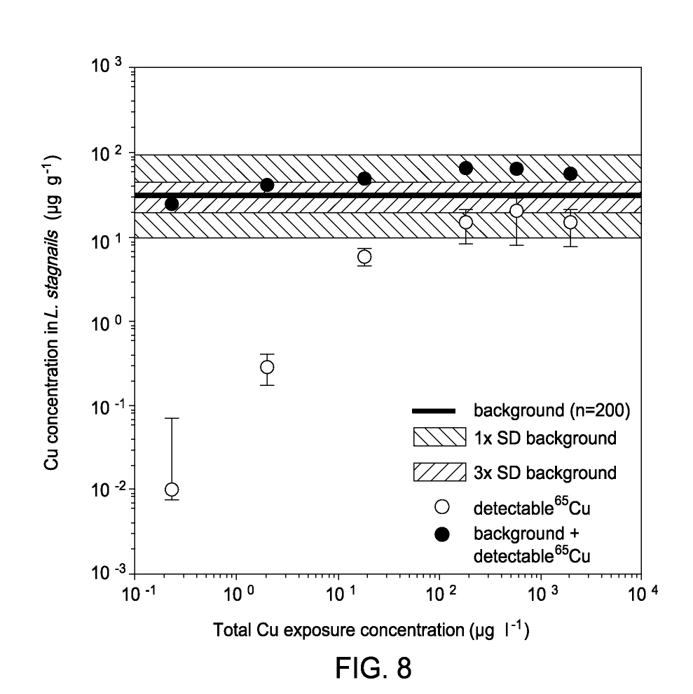

To determine whether isotopically enriched ⁶⁵CuO-s NPs enhanced detection of Cu uptake in animal tissues after short waterborne exposures, freshwater snails (Lymnaea stagnalis) were exposed for a period of 24 hrs to different concentrations of ⁶⁵CuO-s NPs in moderately hard synthetic water. Copper concentrations in soft tissues were then inferred from each Cu isotope using ICP-MS. The net uptake of ⁶⁵Cu in L. stagnalis (open circles, FIG. 8) was linear over a wide range of concentrations that encompassed most environmental exposures; <150 ppb. In the absence of an isotopic label, Cu accumulation was not detectable until a much higher exposure concentration (solid circles, FIG. 8). The effect of the high background Cu concentration (34 μg⁻¹ in L. stagnalis) was circumvented by using isotopically modified NPs, which enhances the detection sensitivity down to environmentally relevant concentrations.

Certain embodiments and features have been described using a set of numerical upper limits and a set of numerical lower limits. It should be appreciated that ranges from any lower limit to any upper limit are contemplated unless otherwise indicated. Certain lower limits, upper limits and ranges appear in one or more claims below. All numerical values are “about” or “approximately” the indicated value, and take into account experimental error and variations that would be expected by a person having ordinary skill in the art.

Various terms have been defined above. To the extent a term used in a claim is not defined above, it should be given the broadest definition persons in the pertinent art have given that term as reflected in at least one printed publication or issued patent. Furthermore, all patents, test procedures, and other documents cited in this application are fully incorporated by reference to the extent such disclosure is not inconsistent with this application and for all jurisdictions in which such incorporation is permitted.

While the foregoing is directed to embodiments of the present invention, other and further embodiments of the invention may be devised without departing from the basic scope thereof, and the scope thereof is determined by the claims that follow.

What is claimed is:

1. A nanoparticle comprising at least one isotopically enriched metal oxide, wherein the metal is copper or zinc.

2. The nanoparticle of claim 1, wherein the at least one isotopically enriched metal oxide is ⁶⁷ZnO or ⁶⁵CuO.

3. The nanoparticle of claim 1, wherein the isotope enrichment ranges from about 90% to about 99% for copper and from about 70% to about 95% for zinc.

4. The nanoparticle of claim 1, wherein the nanoparticle is a sphere, a rod, or spindle shaped.

5. The nanoparticle of claim 1, wherein the nanoparticle consists essentially of the isotopically enriched metal oxide.

6. The nanoparticle of claim 1, wherein an average particle size of the nanoparticle is about 1 nm to about 1,000 nm.

7. The nanoparticle of claim 6, wherein the average particle size of the nanoparticle is about 1 nm to about 100 nm.

8. A composition comprising more than one nanoparticle of claim 1.

9. The composition according to claim 8, wherein the nanoparticles are in the form of a powder or a suspension.

10. The composition according to claim 8, wherein the nanoparticles are in the foam of a colloidal suspension.

11. A method for making the nanoparticle of claim 1, comprising:
converting a precursor of the at least one isotopically enriched metal oxide to the nanoparticle comprising the at least one isotopically enriched metal oxide.

12. A method for making the nanoparticle of claim 1, comprising:
forming a precursor of the nanoparticle, wherein the precursor is an isotopically enriched precursor; and
converting the precursor to the nanoparticle.

13. The method of claim 11, wherein the precursor is converted by thermal decomposition or hydrolysis.

14. The method of claim 11, wherein the precursor is isotopically enriched zinc acetate or isotopically enriched copper chloride.

15. The method of claim 11, wherein the precursor is isotopically enriched copper chloride, and wherein the precursor is dissolved in water and combined with a base.

16. The method of claim 11, wherein the precursor is isotopically enriched copper chloride, wherein the precursor is dissolved in water and combined with a base to form a precipitate, and wherein the precipitate is separated and washed.

17. A method for determining an uptake and extent to which nanoparticles have been distributed within a biological material, comprising:
introducing a composition comprising more than one nanoparticle to a biological material, wherein the nanoparticles comprise an isotopically enriched metal oxide, and wherein the metal is copper or zinc; and
determining a distribution of the nanoparticles in the biological material.

18. The method of claim 17, wherein the nanoparticles consist essentially of the isotopically enriched metal oxide.

19. The method of claim 17, wherein the nanoparticles consist of the isotopically enriched metal oxide.

20. A method of claim 17, wherein the composition is introduced to the biological material by intravenous administration; oral administration; dermal application; direct injection into at least one of: muscle, skin, and peritoneal cavity; or any combination thereof.
